(19) United States
(12) Patent Application Publication
(10) Pub. No.: US 2014/0357978 A1
(43) Pub. Date: Dec. 4, 2014

(54) Computer Based Method for Determining the Size of an Object in an Image

(71) Applicants: Akshay Pai, Valby (DK); Lauge Sorensen, Copenhagen (DK); Erik B. Dam, Bronshoj (DK); Martin Lillholm, Niva (DK); Mads Nielsen, Dragor (DK)

(72) Inventors: Akshay Pai, Valby (DK); Lauge Sorensen, Copenhagen (DK); Erik B. Dam, Bronshoj (DK); Martin Lillholm, Niva (DK); Mads Nielsen, Dragor (DK)

(21) Appl. No.: 13/909,666

(22) Filed: Jun. 4, 2013

(51) Int. Cl. A61B 5/00; A61B 6/00; A61B 5/055; A61B 5/107; A61B 6/03

(52) U.S. Cl. CPC A61B 5/0042; A61B 5/1079; A61B 6/037; A61B 6/032; A61B 5/4842; A61B 5/4848; A61B 5/055; A61B 6/501
USPC 600/410; 600/587; 600/425

(57) ABSTRACT

Volume changes in a brain, brain ventricle, or hippocampus are quantitatively extracted from a comparison of 3D before and after images using an algorithm that distorts a triangulated surface of the before image to produce the surface of the after image and calculates the volume change from the area change.

### BACKGROUND OF THE INVENTION

1. Field of the Invention

The present invention relates to computer analysis of images to extract information regarding changes in the size of an object appearing in the images. The invention has particular but not exclusive relevance to determining changes in the size of the brain and regions thereof appearing in MRI images. Thus the invention has relevance to determining the amount of atrophy as measured by comparing the size of imaged objects in images of the same object obtained at different times.

2. Description of the Prior Art

Brain atrophy estimated from magnetic resonance imaging (MRI) has proven to be a good surrogate marker for the traditional psychometric test for the diagnosis of Alzheimer's disease (AD) (1). Regional analysis of successive MRIs is an intuitive way of computing atrophy. Manual delineations tend to be labor intensive, error prone, and can have low reproducibility (2). Some automated methods, like boundary shift integral, identify atrophy as a shift in the segmentation boundaries, and have shown to perform reliably compared to manual segmentations alone (3). Such approaches are can be limited by relying on strong localization of regions of interest (ROI) and suffers from interpolation effects when the images are non-linearly aligned.

The morphological changes between two images may be determined as the deformation field obtained by non-rigid registration, but extracting this information consistently and accurately remains a challenge. A popular method for estimating the morphological change is Jacobian integration (JI) (4). JI provides localized change maps but lacks numerical precision due to the approximation of the integral by a finite sum. To achieve a better numerical precision, volumetric meshing (VM) approaches have been proposed. While this method provides the necessary accuracy, it suffers from high computational complexity.

### SUMMARY OF THE INVENTION

The present invention now provides a method of computer analysis of images to extract therefrom a quantitative estimate of a change in the volume of an object appearing in said images, said images being separated in time, the method comprising:

a) inputting digital image data of a first and a second 3D image into a computer;

b) in said computer registering the images to align an object appearing in both the first and the second images;

c) applying an algorithm in said computer to the aligned images to extract a quantitative estimate of the difference in the volume of the object shown in the second image compared to the object shown in the first image wherein said algorithm:
i) divides a surface of the object in said first image into faces of unit cuboids;
ii) estimates the change to said faces of cuboids needed to reproduce the surface of the object as seen in the second image; and
iii) calculates the change in the volume of the object as between the first and second images from an sum of the changes in said faces.

The cuboids are preferably cubes and generally will be voxels of the image.

Algorithms for registration of images to align objects common to the images are known in the art, although a suitable procedure is described in detail below.

Optionally, the object is an object in a tomographic image. The invention may for instance be applied to images obtained by a technique selected from the group consisting of Computed Tomography (CT), Positron emission tomography (PET), Single Photon Emission Computed Tomography (SPECT), and creation of a 3D image by assembly from 2D slice images of a physical object.

The latter might be for instance 2D images collected as digital or digitised photographs of histological slicings.

Optionally, the object is a brain or a portion of a brain such as a brain ventricle or a hippocampus. However, the invention is not limited in this regard and the object might be anything for which quantitative determination of change in size would be of interest, including a tumor, a cell, or an organ, whether human or animal organ (in each case, whether to monitor growth or shrinkage).

Optionally, a rate of change with time of said object volume is determined and is compared with standard values relating to a scale of disease progression severity to obtain an estimate of disease severity represented by said first and second images. Where the object is brain or a portion thereof for instance, the disease may be Alzheimer's disease.

Preferably, in said computer by the use of said algorithm, each said first image face is divided into triangles and the triangles are deformed to reproduce the surface in the second image and the computer calculates the change in the volume of the object as between the first and second images from a sum of the changes in said triangles.

Preferably, the computer using said algorithm calculates the change in the volume of the object according to the equation:

|g(Ω)|=∫ₛ_{^{(S)}}xnₓda≈Σᵢ₌₁^{N}g(xᵢ)Aₓg(tᵢ)

wherein Ω is a region of interest of the object in the first image having a surface S, g(Ω) is a transformed region of interest Ω having a transformed surface g(S) relating to the object in the second image, |g(Ω)| is the volume of the transformed region of interest, x is the value of the x-coordinate in an (x,y,z) coordinate system, nₓ is the x-component of the normal of the surface of the region of interest, and for the discrete approximation by taking the areas of triangles xᵢ is the x-component of the barycentre of an i^{th} individual triangle tᵢ and Aₓ is the operator projection to the y-z-plane.

The invention includes a method of evaluating an intervention for effectiveness in treating or preventing Alzheimer's disease comprising obtaining a first brain MRI from each person in a patient group, treating the patient group with said intervention, obtaining a second brain MRI for each patient in said patient group, for each patient extracting from said first and second images a quantitative estimate of a change in the volume of a brain object appearing in said images by a method as described, and scoring the intervention for effectiveness according to said quantitative estimates determined for the patient group.

More generally, the invention includes a method of evaluating an intervention for effectiveness in treating or preventing a pathology associated with change of size of an object in the human or animal body comprising obtaining a first 3D image of said object from each member in a patient group, treating the patient group with said intervention, obtaining a second 3D image for each patient in said patient group, for each patient extracting from said first and second images a quantitative estimate of a change in the volume of said object appearing in said images by a method as described, and scoring the intervention for effectiveness according to said quantitative estimates determined for the patient group.

The intervention may typically be a pharmaceutical treatment but it may also be a change in diet or exercise, or by physiotherapy or the like.

In such a method typically the quantitative estimates obtained for the patient group undergoing said treatment is compared with quantitative estimates obtained from a second patient group treated with a placebo, or with a comparator treatment, or left untreated, between obtaining first and second images.

The method of the invention can provide, at least in preferred embodiments, the local properties of JI and the numerical precision of VM but with a greater computational simplicity than VM in computing volume change from the deformation field obtained from a non-rigid image registration.

As described below, a method we term ‘cube propagation’ (CP), is a simplified mesh based approach to estimate volume changes accurately. We present below a comparison of the numerical precision of JI and CP using a synthetic example and later show the performance of both methods on Alzheimer's disease neuroimaging initiative (ADNI) data.

### DETAILED DESCRIPTION OF PREFERRED EMBODIMENTS

### Image Registration

In the examples which follow, baseline and follow-up images are rigidly aligned through a linear registration between the images using a 6 parameter rigid deformation. Normalized mutual information (NMI) was used as the similarity measure (5). Subsequently, the registration is refined with a b-spline deformation (6). The non-linear registration is driven by a cost function that finds a mapping from the moving image, I, to the reference image, R, optimizing the objective function F:

F=M(I·φ,R)+λP(φ), (Eq. 1)

where M is the similarity measure, λ is a user specified positive constant, P is the regularization term, and φ is the deformation field. The regularization is based on the discrete laplacian in the spline's controls points (φ,j,k), given by,

P=Σ_{i,j,k}|φ_{i,j,k}|², (Eq. 2)

Using a second order term, makes the regularization unbiased in first order transformations (affine image deformations) and thereby unbiased with respect to global atrophy. The final registration is a composition of multiple transformations because this enables modeling larger deformations efficiently. A limited memory BroydenFletcher-Goldfarb-Shanno (L-BFGS) scheme (7) is used for optimization.

### Atrophy Computations

A third order b-spline is used to model the transformation g: [symbol] ³→ [symbol] ³:

(u,v,w)=g(x,y,z)Σᵢ₌₀³Σⱼ₌₀³Σₖ₌₀³Bᵢ(x)Bᵢ(y)Bᵢ(z)φ_{i,j,k} (Eq. 3)

which maps the position (x, y, z) in the moving image I to the position (u, v, w) in the reference image R. Indices i, j, k reference the neighboring control points of the position (x, y, z), B is the basis function, and φ_{i,j,k} are the control point b-spline's coefficients. The first order structure of g relates to the mapping's local expansion or contraction. The purpose of illustrating the benefit of the invention, we compare two ways of estimating regional volume changes: JI and CP.

### Jacobian Integration

Given a region of interest Ω and its volume |Ω|, the volume of the transformed region |Ω(Ω)| is given as:

|g(Ω)|∫_{Ω}|∇g(x,y,z)|dxdydz (4)

where J=≡g is the Jacobian, J: [symbol] ³→ [symbol] ^{3χ3}, and |•| denotes the determinant. Since J1=J1∥J2|, the composed volume change of n warps may be computed as:

∫_{Ω}|J|dxdydz=∫_{Ω}|J1∥J2| . . . |Jn|dxdydz (Eq. 5)

where |Jn| is the Jacobian determinant of the nth warp. Solving the integral analytically is complicated, but may be approximated as a Riemannian sum,

$${\int }_{Ω}^{ } \vert J \vert d x d y d z \approx \frac{1}{{h}^{3}} {\sum }_{( i , j , k ) h ∈ Ω}^{ } \vert J ( ih , jh , kh ) \qquad ( Eq . 6 )$$

P where h is the sampling distance and (i, j, k)εZ³.

### Cube Propagation

The invention seeks to improve the estimate of (Eq. 5) by CP. Volume computation using ST is generally calculated by connecting the vertices of voxel's surfaces to form triangles on the surface and also filling the space with tetrahedra constructed by joining each triangle vertex with the voxel centroid. The accuracy of this method to a certain extent depends on the shape of the deformed voxel. The volume of a cube will be overestimated when the deformation takes any edge to pass through the surface between the centroid and a triangle vertex (8). Similar problems in volume estimation can be overcome by using Gauss' theorem which relates surface integrals to the volume of an object.

Let us define the surface of the region of interest S=∂Ω assuming sufficient smoothness of Ω. Assume a smooth vector field F: [symbol] ³→ [symbol] ³:, then Gauss' theorem relate the closed surface integral to the volume integral:

∫ₛF·nda=∫_{Ω}∇·Fda, (Eq. 7)

where n=(nₓ,n_{y},n_{z}) is the (outwards) surface unit normal. In the following, we shall develop the theory based on the example of an axis-aligned flow: Define F=(x, 0, 0), then ∇·F=1 and

F·n=xnₓ (Eq. 8)

and thereby (Eq. 7) reads

∫_{S}xnₓda=∫_{Ω}1dv=|Ω| (Eq. 9)

This we shall use in the discrete approximation to evaluate the volume of g(Ω). Assume a triangulated closed surface S=t₁, t₂, . . . , t_{N} (later we will assume Ω to be a collection of unit voxels and S to be the triangulated voxel surfaces). Recalling the area of a triangle t projected down to the y-z plane is given as

Aₓ(t)=Anₓ (Eq. 10)

we obtain

∫_{S}xnₓda=Σᵢ₌₁^{N}xᵢAₓ(tᵢ) (Eq. 11)

where xᵢ is the x-coordinate of the centroid of the ith triangle tᵢ. This is exact and not an approximate when S is a collection of plane triangles. Now, we substitute the transformed volume into (Eq. 9) and obtain:

|g(Ω)|=∫_{g}_{^{(S)}}xnₓda≈Σᵢ₌₁^{N}g(xᵢ)Aₓg(tᵢ) (Eq. 11a)

Here the approximation comes from the fact that g is non-linear and the transformed triangles thereby are only a locally linear approximation of the deformation. By notation g(tᵢ) we denote the planar triangle obtained by transforming the corners of tᵢ.

Assuming Ω to be a collection of voxels, where each of the six faces may be triangulated into two triangles, one may evaluate JI by summing the transformation determinant in all the voxel centers or by summing the x-coordinate of the transformed surface triangle centroids weighted by the x-component of the transformed triangle normal. The latter may be performed for all voxels, creating a voxel-wise change-map for later summation, or for only the surface of a given region. It may also be stored for all voxel faces for later indexing for a specific ROI.

Normally, we shall assume that the surface integration in the cube propagation is more numerically precise than the volume integration in the JI due to the fact that only approximation is made on the surface, and not at the interior, and the fact that the JI depends on the first order properties of the deformation g. This will normally vary faster than g itself which the cube propagation is dependent on. This will be tested in below.

### BIAS

A recent study by (9) showed significant bias in atrophy computations when the registrations are unidirectional. This was due to the different blurring applied to the moving image during interpolation. Various bidirectionality schemes have been tested (10). Here, we follow a pairwise symmetric registration scheme where the atrophy scores (AS) are computed as follows,

$$AS = exp ( \frac{log ( {\int }_{{Ω}_{R}}^{ } {V}_{R} d x d y d z ) - log ( {\int }_{{Ω}_{I}}^{ } {V}_{I} d x d y d z )}{2} ) \qquad ( Eq . 12 )$$

where Ω is the ROI for quantification, R and I are the baseline and follow-up images respectively and V is the voxel-wise volume change.

### Experiments and Results

### Synthetic

To test the accuracy and robustness of CP and JI, we have constructed synthetic deformations of a cube sheared at constant volume. This is intended to mimic a relatively extreme but likely brain degeneration: some nestled regions that undergo structural changes while the volume remains constant. The deformed images were constructed as follows: A cube of size 10 mm³ is sheared several times and the non-rigid registration framework described in the previous sections is applied on it. A regularization constant of 1.0 was empirically chosen. For simplicity, the cube is sheared in one direction only. CP is found to be fairly consistent and unbiased over changes in the structure with constant volume but when using JI the error increases with the increase of deformity.

### Application to ADNI Data

### Brain Data and Preprocessing

Data used in this section was obtained from the ADNI database (www.adni-info.org/). Baseline and 12 month follow-up 1.5 T 3D T1-weighted scans of whole brains were analyzed for a subset of patients from the ADNI database. This included 24 normal controls (NC) and 48 Alzheimer's disease (AD) patients (11). Freesurfer pipeline (12) was used to correct for bias and generate segmentations. The images are given in 2563 isotropic voxel cubes, where each voxel is 1 mm³ cube. The segmentations are later used to define the ROI.

### Selection of Evaluation Points

A sparse set of points are sufficient to compute NMI at the coarser levels of transformation. However, at the finest level of transformation, where maximum image information is available, a higher number of evaluation points need to be used. In this example, we use dense sampling (every voxel) in a band along the cerebral boundaries and hippocampus together with coarser sampled points over the rest of the image as evaluation points in the final transformation. The band is generated by the following morphological operations.

E=Iₛ⊕(B⊕B)−Iₛ⊖B (Eq. 13)

where B is the structuring element (3-dimensional sphere of radius 6 mm), ⊖ is morphological dilation, and ⊕ is morphological erosion. Iₛ is the binary image consisting of the brain segmentations (minus ventricles, cerebrospinal fluid (CSF) and brain stem). Ventricles are not considered because they have stronger image forces, hence, a coarser sampling should suffice. In addition, since hippocampus is a very important biomarker in AD, a dense sampling is also considered in a box of 5 voxels beyond the boundaries of the hippocampus.

### Parameters

3 and 5 levels of transformation are used in the rigid and non-rigid registration respectively. For the first 4 levels of non-rigid registration and all levels of the rigid registration, the evaluation points are sampled at every 3rd voxel. On the finest level of registration, the sampling follows Section 5.2.2 in union with sampling at every 3rd voxel over the rest of the image. Different Gaussian blurring is applied to both the images at different levels of registration (kernel sizes of 15, 8, 2 mm for rigid registration and 2.0, 1.5, 1.5, 1.5, 0.2 mm for the non-rigid registration). A regularization constant of 0.03 was empirically chosen.

### Quantification

Effect sizes (Cohen's d) is used to quantify the efficacy of both the approaches which is given by,

$$d = \frac{{μ}_{AD} - {μ}_{NC}}{\sqrt{\frac{{σ}_{AD}^{2} + {σ}_{NC}^{2}}{2}}}$$

where μ_{AD} and μ_{NC} are means of AD and NC atrophy measures respectively and σ_{AD} and σ_{NC} are the respective standard deviations.

TABLE 1
Cohen's d for atrophy computations using JI and CP.

| | JI | CP |
|---|---|---|
| Whole Brain | 0.83 | 0.95 |
| Ventricles | 1.05 | 1.19 |
| Hippocampus | 1.52 | 1.55 |

TABLE 2
Mean and standard deviation of atrophy measures in different
structures for different cognitive groups using JI and CP.

| | JI | CP |
|---|---|---|
| Whole Brain | NC = −0.45 ± 0.92 | NC = −0.45 ± 0.92 |
| | AD = −1.39 ± 1.32 | AD = −1.55 ± 1.35 |
| Ventricles | NC = 4.84 ± 5.31 | NC = 4.81 ± 5.28 |
| | AD = 11.12 ± 6.58 | AD = 11.49 ± 5.93 |
| Hippocampus | NC = −0.78 ± 1.43 | NC = −0.77 ± 1.40 |
| | AD = −3.59 ± 2.18 | AD = −3.57 ± 2.12 |

It can be seen in Table 1 that CP outperforms JI in atrophy computations of whole brain and ventricles. Both methods perform fairly similar in hippocampus atrophy computation. Table 2 presents the mean and standard deviation of AD and NC groups measured by JI and CP.

### Scan Rescan Data

We also evaluated the scan rescan reproducibility of JI and CP on 9 subjects that were scanned and rescanned. Both JI and CP produce similar results with standard error of the mean (SEM) less than 1.0% in the hippocampus, whole brain and ventricles.

### Evaluation of the Results

The primary advantage of using voxel based atrophy computation schemes is that they provide localized atrophy maps. It was observed that computing the volume change in JI as a Riemannian approximation leaves an inaccuracy of order h, where h is the sampling distance. While the inaccuracy left in CP is due to the linear interpolation at the outer surface only, and this relates to second order changes of the deformation field and thereby to h². One advantage of this triangulation method is its symmetry. The construction is such that, neighboring voxels share triangles and no volume can escape in between voxels. In addition to capturing volume changes, the underlying atrophy computation methods should measure no change accurately too. CP is fairly insensitive to structural changes of an object (with constant volume), while JI become increasingly inaccurate with complicated deformations. This effect can be seen in the whole brain and ventricle results. The presence of CSF can make the deformations complicated. However, both methods perform similarly in the measurement of atrophy in the hippocampus as seen in Table 2. Similarly the mean and standard deviations, of both JI and CP, in the NC group are fairly similar. The similarity in the methods is also observed in scan-rescan studies where the interval between the scans is too short for any physiological changes to happen.

The cube propagation method presented here enables a very precise quantification of volume changes using the deformation field of a non-rigid registration. The method's reliability on real data is proven through the application on brain data. Both the methods demonstrated a similar performance in measuring volume changes in the NC group and hippocampus in the AD group. However, CP outperforms JI while measuring volume changes in whole brain and ventricles. Although the focus of the example was on Alzheimer's disease, where the deformations are smaller in the considered time interval, this method is also applicable in scenarios with relatively larger deformations.

More generally, the illustrated method may be applied to 3D images generally, such as tomograms computed from x-ray absorption, phase, diffraction, electron microscopy, single particles, histological slicings, or nuclear imaging in terms of positron emission tomograms or single photon emission computed tomography and to images of any object or part of an object. Other relevant fields of use therefore include quantitative determination of tumor growth or shrinkage, cell growth, animal organ growth.

The following references are cited herein.
1. Fox N C, Black R S, Gilman S, Rossor M N, Griffith S G, Jenkins L, and Koller M; AN1792(QS-21)-201 Study. Effects of a beta immunization (an 1792) on MRI measures of cerebral volume in Alzheimer's disease. Neurology, 64(9):1563-1572, May 2005.
2. Barnes J, Foster J, Boyes R G, Pepple T, Moore E K, Schott J M, Frost C, Scahill R I, and Fox N C. A comparison of methods for the automated calculation of volumes and atrophy rates in the hippocampus. Neuroimage, 40(4):1655-1671, January 2008.
3. Leung K K, Clarkson M J, Bartlett J W, Clegg S, Jack C R Jr, Weiner M W, Fox N C, Ourselin S, and Alzheimer's Disease Neuroimaging Initiative. Robust atrophy rate measurement in Alzheimer's disease using multi-site serial MRI: Tissue-specific intensity normalization and parameter selection. Neuroimage, 50(2):516-523, April 2010.
4. Boyes R G, Rueckert D, Aljabar P, Whitwell J, Schott J M, Hill D L, and Fox N C. Cerebral atrophy measurements using Jacobian integration: Comparison with the boundary shift integral. Neuroimage, 32(1):159-169, August 2006.
5. Sune Darkner and Jon Sporring. Locally orderless registration. IEEE Transactions on Pattern Analysis and Machine Intelligence, 2012. (PrePrint).
6. Rueckert D, Sonoda L I, Hayes C, Hill D L G, Leach M O, and Hawkes D J. Non-rigid registration using free-form deformations: Application to breast MRI images. IEEE Transactions on Medical Imaging, 18(8):712-721, 1999.
7. Dong C. Liu, Jorge Nocedal, Dong C. Liu, and Jorge Nocedal. On the limited memory BFGS method for large scale optimization. Mathematical Programming, 45:503-528, 1989.
8. Hughes S W, D'Arcy T J, Maxwell D J, Saunders J E, Ruff C F, Chiu W S, and Sheppard R J. Application of a new discreet form of Gauss' theorem for measuring volume. Physics in medicine and biology, 41(9):1809-1821, September 1996.
9. Paul A. Yushkevich, Brian B. Avants, Sandhitsu R. Das, John Pluta, Murat Altinay, Caryne Craige, and the Alzheimer's Disease Neuroimaging Initiative. Bias in estimation of hippocampal atrophy using deformation based morphometry arises from asymmetric global bias in estimation of hippocampal atrophy using deformation-based morphometry arises from asymmetric global normalization: An illustration in adni 3 tesla MRI data. Neuroimage, 50(2):434-445, April 2010.
10. Kelvin K. Leung, Gerard R. Ridgway, Sebastien Ourselin, Nick C. Fox, and The Alzheimer's Disease Neuroimaging Initiative. Consistent multi-time-point brain atrophy estimation from the boundary shift integral. Neuroimage, 59:3995-4005, 2012.
11. Katherine Chong, Wan Chi Lau, Jason Leong, Joyce Suhy, and Joonmi Oh. Longitudinal volumetric mri analysis for use in Alzheimer's disease multi-site clinical trials: Comparison to analysis methods used in ADNI and correlation to mmse change. volume 6, 2010.
12. M Reuter, H. D Rosas, and B Fischl. Highly accurate inverse consistent registration: A robust approach. Neuroimage, 53(4):1181-1196, 2010.

In this specification, unless expressly otherwise indicated, the word ‘or’ is used in the sense of an operator that returns a true value when either or both of the stated conditions is met, as opposed to the operator ‘exclusive or’ which requires that only one of the conditions is met. The word ‘comprising’ is used in the sense of ‘including’ rather than in to mean ‘consisting of’. All prior teachings acknowledged above are hereby incorporated by reference. No acknowledgement of any prior published document herein should be taken to be an admission or representation that the teaching thereof was common general knowledge anywhere at the date hereof.

What is claimed is:

1. A method of computer analysis of images to extract therefrom a quantitative estimate of a change in the volume of an object appearing in said images, said images being separated in time, the method comprising:
a) inputting digital image data of a first and a second 3D image into a computer;
b) in said computer registering the images to align an object appearing in both the first and the second images;
c) applying an algorithm in said computer to the aligned images to extract a quantitative estimate of the difference in the volume of the object shown in the second image compared to the object shown in the first image wherein said algorithm:
i) divides a surface of the object in said first image into faces of unit cuboids;
ii) estimates the change to said faces of cuboids needed to reproduce the surface of the object as seen in the second image; and
iii) calculates the change in the volume of the object as between the first and second images from an sum of the changes in said faces.

2. A method as claimed in claim 1, wherein the object is an object in a tomographic image.

3. A method as claimed in claim 2, wherein the object is an object in an image obtained by a technique selected from the group consisting of Computed Tomography (CT), Positron emission tomography (PET), Single Photon Emission Computed Tomography (SPECT), and creation of a 3D image by assembly from 2D slice images of a physical object.

4. A method as claimed in claim 1, wherein the object is a brain or a portion of a brain.

5. A method as claimed in claim 4, wherein the object is a brain ventricle or a hippocampus.

6. A method as claimed in claim 1, wherein a rate of change with time of said object volume is determined and is compared with standard values relating to a scale of disease progression severity to obtain an estimate of disease severity represented by said first and second images.

7. A method as claimed in claim 1, wherein, in said computer, each said first image face is divided into triangles and the triangles are deformed to reproduce the surface in the second image and the computer calculates the change in the volume of the object as between the first and second images from a sum of the changes in said triangles.

8. A method as claimed in claim 7, wherein the computer calculates the change in the volume of the object according to the equation:

|g(Ω)|=∫ₛ_{^{(S)}}xnₓda≈Σᵢ₌₁^{N}g(xᵢ)Aₓg(tᵢ)
wherein Ω is a region of interest of the object in the first image having a surface S, g(Ω) is a transformed region of interest Ω having a transformed surface g(S) relating to the object in the second image, |g(Ω)| is the volume of the transformed region of interest, x is the value of the x-coordinate in an (x,y,z) coordinate system, nₓ is the x-component of the normal of the surface of the region of interest, and for the discrete approximation by taking the areas of triangles xᵢ is the x-component of the barycentre of an i^{th} individual triangle tᵢ and Aₓ is the operator projection to the y-z-plane.

9. A method of evaluating an intervention for effectiveness in treating or preventing Alzheimer's disease comprising:
obtaining a first brain MRI from each person in a patient group, treating the patient group with said intervention,
obtaining a second brain MRI for each patient in said patient group,
for each patient extracting from said first and second images a quantitative estimate of a change in the volume of a brain object appearing in said images by the method as claimed in claim 1, and
scoring the intervention for effectiveness according to said quantitative estimates determined for the patient group.

10. A method as claimed in claim 9, wherein the intervention is a pharmaceutical treatment.

11. A method as claimed in claim 9, wherein the quantitative estimates obtained for the patient group undergoing said treatment is compared with quantitative estimates obtained from a second patient group treated with a placebo, or with a comparator treatment, or left untreated between obtaining first and second images.
